(19) United States
(12) Patent Application Publication
(10) Pub. No.: US 2019/0000808 A1
(43) Pub. Date: Jan. 3, 2019

(54) COMPOSITION AND METHODS FOR THE TREATMENT OF BLEPHOPHAROPTOSIS

(71) Applicant: Allergan, Inc., Irvine, CA (US)

(72) Inventors: Charles F. BOSWORTH, Oceanside, CA (US); Houman D. HEMMATI, Los Angeles, CA (US); David A. HOLLANDER, Tustin, CA (US)

(21) Appl. No.: 15/744,017

(22) PCT Filed: Jul. 6, 2016
(86) PCT No.: PCT/US2016/041367

§ 371 Date: Jan. 11, 2018

Related U.S. Application Data
(60) Provisional application No. 62/191,951, filed on Jul. 13, 2015.

(51) Int. Cl. A61K 31/4178; A61K 31/4174; A61P 25/02; A61K 9/00

(52) U.S. Cl. CPC A61K 31/4178; A61K 9/0014; A61P 25/02; A61K 31/4174

(57) ABSTRACT

The present invention relates to compositions and methods for the treatment and prevention of ptosis and more specifically to compositions and methods for the non-surgical treatment and prevention of ptosis and methods for the improvement of night vision, administering a combination of pilocarpine and oxymetazoline.

This application claims priority to U.S. Provisional Patent Application No. 62/191,951, filed Jul. 13, 2015, the entire content of which is incorporated herein by reference.

### FIELD OF THE INVENTION

The present invention relates generally to compositions and methods for the treatment and prevention of blepharoptosis (“ptosis”) and more specifically to compositions and methods for the non-surgical treatment, prevention and amelioration of ptosis. The present invention also relates for compositions and methods for the improvement of night vision.

### BACKGROUND OF THE INVENTION

### Blepharoptosis

Blepharoptosis, more commonly referred to as ptosis, refers to the drooping or inferodisplacement of the upper eyelid below the normal adult upper eyelid margin approximately 0.5-2 mm below the superior corneal limbus. AAO (2007). Orbit, Eyelids, and Lacrimal System. Singapore, American Academy of Ophthalmology. As ptosis intrudes on the optical axis of the eye it can shift from an isolated problem that is merely cosmetic in nature to a condition that obstructs vision. Ptosis may be due to a myogenic, neurogenic, aponeurotic, mechanical or traumatic causes. Usually, ptosis occurs in isolation, but may be associated with various medical conditions such as immunological, degenerative, neurological, hereditary disorders, tumors, or infections. Ptosis can either be present at birth (congenital ptosis) or develop later in life (acquired ptosis). Acquired ptosis can have a myogenic, neurogenic (lesion of the sympathetic nerves, peripheral or central lesion of the oculomotor nerve), aponeurotic, traumatic or a mechanical, cause.

The most common type of ptosis is the acquired type. Acquired ptosis is more common in the elderly, can affect one or both eyes, and is typically caused by stretching or disinsertion of the aponeurosis of the levator palpebrae superioris (levator) muscle. The upper lid retractors are a group of muscles whose main function is the keep the upper eyelid elevated by inserting on the superior margin of the tarsus, the dense fibrous tissue that maintains the structural integrity of the eyelids. The primary muscle in this group is the levator, and the secondary muscle in this group is Muller's muscle.

The levator palpebrae superioris is a striated muscle that is innervated by the superior division of the oculomotor nerve (cranial nerve III) and controls opening of the eyelid. The muscle originates just above the annulus of Zinn along the lesser wing of the sphenoid and is roughly 40 mm in length. The muscle continues anteriorly and at the equator of the globe, a transition from muscle to an aponeurosis occurs, approximately 15-17 mm from the superior tarsal border. At approximately the level of the upper border of the tarsus, the levator/aponeurosis complex sends attachments to the skin forming the upper eyelid crease. The entire levator/aponeurosis complex is approximately 54-60 mm in length.

The orbital septum connects from the arcus marginalis of the frontal bone and blends with the levator aponeurosis 5-20 mm above the superior tarsal border and acts as a barrier in the upper eyelid. The orbital septum varies with each individual anatomically and can be thick or thin. Surgically, the orbital septum is an important structure with ptosis.

Beneath the levator/aponeurosis complex is Muller's muscle. Muller's muscle is attached to the levator/aponeurosis complex and inserts by a 0.5-1 mm-long tendon into the superior tarsal plate. Muller's muscle is sympathetically innervated and regulates the palpebral fissure width.

Clinically ptosis can be categorized in severity based upon the amount the upper eyelid droops: minimal drooping (1-2 mm), moderate drooping (3-4mm), or severe drooping (>4 mm) entirely covering the pupil. Patients with ptosis often complain about a tired appearance, blurred vision, and increased tearing. Patients with significant ptosis may need to tilt their head back into a chin-up position, lift their eyelid with a finger, or raise their eyebrows to perform normal visual tasks. Continuous activation of the forehead and scalp muscles may additionally cause tension headache and eyestrain. If congenital ptosis is not corrected, amblyopia, leading to permanently poor vision, may develop. Finsterer, J. (2003). “Ptosis: causes, presentation, and management.” Aesthetic Plast Surg 27(3): 193-204.

Minimal or moderate dropping is primarily a cosmetic concern, as patients often complain of facial asymmetry and/or a tired-looking appearance. Finsterer, J. (2003). “Ptosis: causes, presentation, and management.” Aesthetic Plast Surg 27(3): 193-204. As the upper eyelid encroaches on the visual axis (≥3 mm), ptosis may have a significant impact on vision impacting the superior visual field as well as restricting central vision if the eyelid encroaches on the pupillary axis.

Ptosis can significantly impact activities of daily living. For example, many ptosis patients complain of difficulty reading because the ptosis is worsened in down gaze. AAO (2007), Orbit, Eyelids, and Lacrimal System. Singapore, American Academy of Ophthalmology. Many ptosis patients also complain of difficulty functioning at night where their pupil diameter is increased to increase light absorption. AAO (2007). Orbit, Eyelids, and Lacrimal System. Singapore, American Academy of Ophthalmology.

Surgical repair of ptosis depends largely on age, etiology, whether one or both eyelids are involved, the severity of ptosis, the function of the levator muscle, and presence of additional ophthalmologic abnormalities. Finsterer, J. (2003). “Ptosis: causes, presentation, and management.” Aesthetic Plast Surg 27(3): 193-204. At the present time, there are no approved pharmacological treatments for ptosis. There is typically no insurance coverage for the surgical repair of mild ptosis because it is considered medically unnecessary for a cosmetic concern. Anderson, R. L. and J. B. Holds (1990). “Does anyone know how to differentiate a ‘functional’ defect from a cosmetic one?” Arch Ophthalmol 108(12): 1685-1686. When ptosis is associated with a functional impact insurance coverage for surgical repair can be obtained. Determination of a functional deficit requires visual field testing.

For minimal ptosis, Muller's muscle conjunctival resection (Mullerectomy) is typically performed, as it is the least invasive of the surgical treatments for ptosis. (Finsterer, J. [2003]. “Ptosis: causes, presentation, and management.” Aesthetic Plast Surg 27(3): 193-204.)(Aakalu, V. K. and P. Setabutr [2011]. “Current ptosis management: a national survey of ASOPRS members.” Ophthal Plast Reconstr Surg 27(4): 270-276.). For moderate ptosis, shortening of the levator muscle or levator advancement are performed. For severe ptosis, a brow/frontalis suspension surgery, a more invasive surgery that involves placement of a permanent implant, is typically performed.

Muller's muscle contains an abundance of alpha-1 adrenergic receptors. Activation of alpha-1 adrenergic receptors on Muller's muscle by adrenergic agonists such as phenylephrine causes eyelid elevation (and, thereby, a temporary reversal of ptosis)(Skibell, B. C., J. H. Harvey, et al. (2007). “Adrenergic receptors in the ptotic human eyelid: correlation with phenylephrine testing and surgical success in ptosis repair.” Ophthal Plast Reconstr Surg 23(5): 367-371. Consistent with this pharmacology, a majority of ophthalmic plastic and reconstructive surgeons instill a single drop of phenylephrine (typically 2.5%) into the eye(s) with a ptotic eyelid to assess whether doing so will temporarily reverse the observed ptosis. Aakalu, V. K. and P. Setabutr (2011). “Current ptosis management: a national survey of ASOPRS members.” Ophthal Plast Reconstr Surg 27(4): 270-276. In patients who exhibit a good response to phenylephrine, mullerectomy is typically successful in treating ptosis. A poor response to phenylephrine is associated with a sub-optimal outcome from mullerectomy. Maheshwari, R. and S. Maheshwari (2011). “Muller's muscle resection for ptosis and relationship with levator and Muller's muscle function.” Orbit 30(3): 150-153. Due to its temporary improvement of ptosis, potential cardiovascular side effects, and induction of pupillary dilation, phenylephrine is neither clinically nor commercially viable as a long-term pharmacological treatment for ptosis.

Surgical management of ptosis is often unsuccessful because the surgery can result in under correction or overcorrection of the ptotic eyelid leaving the patient's eyelids asymmetrical. Surgical management of ptosis often requires more than one surgery because of its progressive nature and the return of eyelid drooping after surgery. Surgical management of ptosis may also result in infection, poor response to local anesthesia, intraoperative bleeding, postoperative bleeding, infections, eyelid crease abnormalities, distortion of the eyelid margin contour, and foreign body sensation. Finsterer, J. (2003). “Ptosis: causes, presentation, and management.” Aesthetic Plast Surg 27(3): 193-204. What is needed is a topical pharmaceutical product for the treatment of ptosis which provides long lasting treatment of ptosis and allows patients to avoid surgery.

### Night Blindness

Night blindness refers to a below-average ability to see at night or in low light. Night blindness is not a disorder in itself, but rather a symptom of an underlying condition. It can occur in people of all ages, even young children. Night blindness is sometimes referred to as nyctalopia or impaired dark adaptation. Night blindness can be congenital or a symptom of a number of conditions that can be acquired such as myopia, cataracts, the side-effect of certain drugs such as diabetes medication(s) or vitamin deficiencies.

Pupillary dilation to low light or dim light is a teleologic adaption to allow more light to enter our eyes to improve vision. In humans, the degree to which pupils will dilate in response to dim light ranges from a maximal dilation in complete darkness from 3 to 9 mm.

When the pupil is very small (ie, ˜1 mm) light will diffract off the iris degrading visual performance. When the pupil is very large (eg, >6 mm) peripheral aberrations in the optics of the eye will degrade visual performance. The best optical performance for the human eye is achieved with a pupil diameter of approximately 2.5 mm. For low light environments maintaining a pupil diameter between 2.5 mm to 6 mm would be ideal. Accordingly, pupil dilation is considered to be “controlled” when a desirable pupil diameter is achieved or maintained in low ambient light situations, as described herein. Pupil dilation control can include a reduction in the size of a pupil, or the maintenance of a desired level of pupil dilation.

There is currently no topical ophthalmic medication to improve visual acuity in low light environments.

Pilocarpine is a muscarinic receptor agonist that mimics the actions of the parasympathetic neurotransmitter, acetylcholine, on smooth muscle. This causes two effects which enhance near vision: 1) constriction of the iris sphincter muscle, resulting in pupil miosis; and 2) constriction of the ciliary muscle, resulting in central lens steepening and lens accommodation (focusing from distance to near) in humans (as well as in animal models). Pilocarpine, has been used as an isolated medication for the treatment of presbyopia and mild hyperopia, but has not been very effective because topical concentrations below 0.5% produce minimal effect in the accommodation of the eye and concentrations above 0.5% are not tolerated due to side effects such as red eyes, ocular pain, brow ache, and headache.

Oxymetazoline is a mixed agonist of α₁a and α₂ adrenergic receptors, with some possible muscarinic activity. Stimulation of adrenergic receptors by the sympathetic neurotransmitter, noradrenaline, causes contraction of the radial (dilator) muscle and subsequent dilation of the pupil. Oxymetazoline's α₁a and muscarinic activity may partially antagonize and delay the onset of action of pilocarpine, potentially reducing the rate and/or severity of brow ache. This delay may also prolong miosis when oxymetazoline and pilocarpine are used in combination, as has been shown in clinical studies. In addition, oxymetazoline may antagonize the pilocarpine effect on ciliary muscle contraction and lens curvature (accommodation), leading to less loss of distance visual acuity with the combination of oxymetazoline and pilocarpine than with pilocarpine alone.

A pharmacological product is needed which can be administered topically to the eyes which will improve visual acuity in patients in low light environments.

### SUMMARY OF THE INVENTION

This present invention is intended to provide a reversible, pharmacologic method for delaying or avoiding the need for ptosis surgery in patients with disruption of the visual axis secondary to ptosis through the following mechanisms:

Elevation of the upper eyelid through stimulation of Muller's muscle by oxymetazoline;

Constriction of the pupil by pilocarpine, which improves both depth of focus and near visual acuity, and draws the pupillary margin away from the ptotic eyelid margin; and,

Prolongation of the aforementioned two effects by the interaction of pilocarpine and oxymetazoline during concomitant exposure.

The present invention provides compositions and methods for the treatment of ptosis by the use of oxymetazoline and pilocarpine formulations. The application also provides compositions and methods to optimize and/or control pupil size or diameter in low light conditions.

Oxymetazoline, C₁₆H₂₄N₂O, also known as 3-(4,5-dihydro-1H-imidazol-2-ylmethyl)-2,4-dimethyl-6-tert-butyl-phenol, has the following structure:

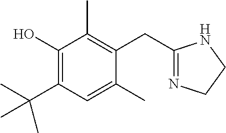 [SMILES: Cc1cc(C(C)(C)C)c(O)c(C)c1CC1=NCCN1]

Oxymetazoline may be used in a salt form, such as oxymetazoline HCl.

Pilocarpine, C₁₁H₁₆N₂O₂, also known as (3S,4R)-3-Ethyl-4((1-methyl-1H-imidazol-5-yl)methyl)dihydrofuran-2(3H)- one, has the following structure:

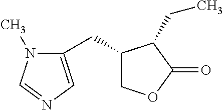

Pilocarpine may be used in a salt form, such as pilocarpine HCl.

Some embodiments of the present invention may include:
1. A method of treating ptosis in a patient suffering therefrom comprising administering a combination of pilocarpine and oxymetazoline.
2. The method of embodiment 1 wherein the pilocarpine and oxymetazoline are administered in a single formulation.
3. The method of embodiments 1 and 2 wherein the composition is administered topically to the eye.
4. The method of treating ptosis comprising administering to a subject in need thereof a composition comprising a therapeutically effective amount of a composition comprising pilocarpine and oxymetazoline.
5. The method of embodiment 4 wherein the pilocarpine is pilocarpine HCl and oxymetazoline is oxymetazoline HCl.
6. The method of embodiments 1 and 5 wherein the composition comprises about 0.125% w/v oxymetazoline and about 1% w/v pilocarpine.
7. The method of embodiments 1 and 4 wherein the composition comprises about 0.1-0.15% w/v oxymetazoline and about 0.05%-0.15% w/v pilocarpine.
8. The method of embodiments 1 and 4 wherein the composition comprises about 0.01-0.2% w/v oxymetazoline and about 0.05-2% w/v pilocarpine.
9. The method of embodiment 7 wherein the composition comprises about 0.0125%-0.125% w/v oxymetazoline and about 0.5-1.5% w/v pilocarpine.
10. The method of embodiment 6 wherein the composition is dosed at least once a day.
11. The method of embodiment 6, wherein the composition is dosed twice a day.
12. The method of embodiment 6, wherein the composition is dosed three times a day.
13. The method of embodiments 8, 9 and 10 wherein the ptosis may be acquired or congenital ptosis.
14. The method of embodiments 8, 9 and 10 wherein the ptosis is myogenic ptosis and the patient is suffering from one selected from the groups consisting of myasthenia gravis, Bell's palsy, myotonic dystrophy, facio-scapulo-humeral muscular dystrophy, oculopharyngeal-muscular dystrophy, congenital myopathies and mitochondriopathy.
15. The method of embodiments 8, 9 and 10 wherein the ptosis is neurogenic ptosis, aponeurotic ptosis, mechanical ptosis, traumatic ptosis, or pseudoptosis.
16. The method of embodiment 8, 9 and 10 wherein the ptosis is congenital ptosis selected from the group consisting of idiopathic ptosis, myogenic ptosis, aponeurotic ptosis, neurogenic ptosis, mechanical ptosis or traumatic ptosis.
17. The method of embodiments 8, 9, 10 and 14 wherein the patient suffers from Homer's Syndrome or Marcus-Gunn Syndrome.
18. The method of embodiment 2 wherein the formulation is prescribed as an alternative to surgery.
19. The method of embodiment 2 wherein the formulation is applied after surgery to treat ptosis.
20. The method of embodiment 2 wherein the formulation is applied before ophthalmic surgery to treat ptosis.
21. The method of embodiment 17 wherein the formulation is applied after surgery to correct ptosis in one or both eyes.
22. The method of embodiments 2, 7 and 8 wherein the administration of the composition results in relief of the symptoms of ptosis.
23. The method of embodiment 21 wherein relief of the symptoms of ptosis includes retraction of the upper eyelid.
24. The method of embodiment 21 wherein relief of the symptoms of ptosis includes retraction of the lower eyelid.
25. The method of embodiment 1 wherein oxymetazoline are pilocarpine are administered concurrently.
26. A composition comprising oxymetazoline and pilocarpine.
27. The composition of embodiment 25 wherein the composition has about 0.01-0.5% w/v oxymetazoline and about 0.5-1.5% w/v pilocarpine.
28. The composition of embodiments 25 and 26 wherein the composition has one or more of the excipients of Tables II and III.
29. The composition of embodiment 25 wherein the composition has about 0.125% w/v oxymetazoline and about 1% w/v pilocarpine.
30. The composition of embodiment 28 wherein the composition has 0.125% w/v and 1% w/v pilocarpine.
31. The composition of embodiments 25, 26, 28 and 29 wherein the compositions also include one or more of the excipients in Tables II and III.
32. Use of oxymetazoline and pilocarpine in a single composition in the manufacture of a medicament for the treatment of ptosis.
33. The use of 0.01-0.5% w/v oxymetazoline and about 0.5-1.5% w/v pilocarpine in a single composition in the manufacture of a medicament for the treatment of ptosis.
34. The use of about 0.125% w/v oxymetazoline and about 0.1% w/v pilocarpine in a single composition in the manufacture of a medicament for the treatment of ptosis.
35. Use of oxymetazoline and pilocarpine for the treatment of ptosis.
36. Use of about 0.01-0.5% w/v oxymetazoline and about 0.5-1.5% w/v pilocarpine for the treatment of ptosis.
37. Use of about 0.125% w/v oxymetazoline and about 0.1% w/v pilocarpine in a single composition in the manufacture of a medicament for the treatment of ptosis.
38. A composition for use in treating blepharoptosis comprising oxymetazoline and pilocarpine.
39. The composition of embodiment 37 wherein the composition comprises from about 0.01-0.2% w/v oxymetazoline and 0.01-2.0% w/v pilocarpine.
40. The composition of embodiment 38 wherein the composition comprises from about 0.05-0.15% w/v oxymetazoline and 0.05-1.5% w/v pilocarpine.
41. The composition of embodiments 37 and 38 wherein the composition comprises about 0.1-0.15% w/v oxymetazoline and 0.05-1.5% w/v pilocarpine.
42. The composition of embodiments 37, 38, 39 and 40 wherein the composition comprises about 0.125% w/v oxymetazoline and about 0.1% w/v pilocarpine.
43. The composition of embodiments 37, 38, 39 and 40 wherein the composition comprises 0.125% w/v oxymetazoline and 1% w/v pilocarpine.
44. The composition of embodiments 37, 38, 39, 40, 41 and 42 wherein the composition is preserved.
45. The composition of embodiment 43 wherein the preservative is benzalkonium chloride.
46. The composition of embodiments 37, 38, 39, 40, 41 and 42 wherein the composition is in a non-preserved unit dose form.
47. The composition of embodiments 37, 38, 39, 40, 41 and 42 wherein the composition is in an ocular implant.
48. A method of controlling pupil dilation in a patient in a low light environment comprising administering a combination of pilocarpine and oxymetazoline.
49. The method of embodiment 47 wherein the pilocarpine and oxymetazoline are administered in a single formulation.
50. The method of embodiments 47 and 48 wherein the composition is administered topically to the eye to improve visual acuity in a low light environment.
51. A method of treating night vision comprising administering to a subject in need thereof a composition comprising a therapeutically effective amount of a composition comprising pilocarpine and oxymetazoline.
52. The method of embodiments 47, 48, 49 and 50 wherein the composition comprises about 0.125% w/v oxymetazoline and about 1% w/v pilocarpine.
53. The method of embodiment 51 wherein the composition comprises from about 0.1-0.15% w/v oxymetazoline and about 0.05%-1.5% w/v pilocarpine.
54. The method of embodiments 47, 48, 49, 50, 51 and 52 wherein the administration of the composition to a patient results in decreased pupil dilation in response to a low light environment as compared to the patient not being administering the composition.
55. A composition for use in improving visual acuity in a low light environment comprising oxymetazoline and pilocarpine.
56. The composition of embodiment 54 wherein the composition comprises about 0.01-0.2% w/v oxymetazoline and about 0.01-5% w/v pilocarpine.
57. The composition of embodiment 55 wherein the composition comprises about 0.05-0.15% w/v oxymetazoline and about 0.05-2% w/v pilocarpine.
58. The composition of embodiment 56 wherein the composition comprises about 0.1-0.15% w/v oxymetazoline and 0.05-1.5% w/v pilocarpine.
59. The composition of embodiments 54, 55, 56 and 57 wherein the composition comprises about 0.125% w/v oxymetazoline and about 1% w/v pilocarpine.
60. The composition of embodiments 54, 55, 56, 57, and 58 wherein the composition comprises 0.125% w/v oxymetazoline and 1% w/v pilocarpine.
61. The composition of embodiments 54, 55, 56, 57, 58 and 59 wherein the composition comprises 0.125% w/v oxymetazoline HCl and 1% w/v pilocarpine HCl.
62. The composition of embodiment 60 wherein the composition is preserved.

### DETAILED DESCRIPTION OF THE INVENTION

Disclosed herein are compositions and methods for treatment of blepharoptosis.

The terms “about” and “approximately equal” are used herein to modify a numerical value and indicate a defined range around that value. If “X” were the value, “about X” or “approximately equal to X” would generally indicate a value from 0.90X to 1.10X. Any reference to “about X” minimally indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.” About may also refer to a number close to the cited number that would result in a bioequivalent therapeutic effect by a regulatory agency such as the FDA or the EMEA.

The terms “active” , “active pharmaceutical ingredient,” and “API” and the like refer to the active ingredient of a drug product. An API is typically a chemical substance or mixture of chemical substances. Such substances are intended to furnish pharmacological activity or other direct effect in the diagnosis, cure, mitigation, treatment or prevention of disease or to affect the structure and function of the body of a subject.

The terms “effective amount,” “therapeutically effective amount” or “pharmaceutically effective amount” refers to that amount of an active agent effective to treat ptosis, including a range of effects, from a detectable amount of improvement to substantial relief of symptoms. The result can be reduction and/or alleviation of the signs, symptoms, or causes of a disease, or any other desired alteration of a biological system. For example, an “effective amount” for therapeutic uses is the amount of the composition comprising an agent as set forth herein required to provide a clinically significant decrease in an ophthalmic disease. For example, for the given aspect (e.g., length of incidence), a therapeutically effective amount will show an increase or decrease of at least 5%, 10%, 15%, 20%, 25%, 40%, 50%, 60%, 75%, 80%, 90%, or 100%. Therapeutic efficacy can also be expressed as “-fold” increase or decrease. For example, a therapeutically effective amount can have at least a 1.2-fold, 1.5-fold, 2-fold, 5-fold, or more effect over a control. An appropriate “effective” amount in any individual case may be determined using techniques, such as a dose escalation study.

“Formulation” and “composition,” are intended to be equivalent and refer to a composition of matter suitable for pharmaceutical use (i.e., producing a therapeutic effect as well as possessing acceptable pharmacokinetic and toxicological properties).

The term “pharmaceutically acceptable” is used as equivalent to physiologically acceptable. In certain embodiments, a pharmaceutically acceptable composition or preparation will include agents for buffering and preservation in storage, and can include buffers and carriers for appropriate delivery, depending on the route of administration.

As used herein, the terms “prevent” and “treat” are not intended to be absolute terms. Treatment can refer to any delay in onset, e.g., reduction in the frequency or severity of symptoms, amelioration of symptoms, improvement in patient comfort, reduction in lid drooping, improvement of vision in low light environments, and the like. The effect of treatment can be compared to an individual or pool of individuals not receiving a given treatment, or to the same patient before, or after cessation of, treatment.

The terms “subject,” “patient,” “individual,” are not intended to be limiting and can be generally interchanged. That is, an individual described as a “patient” does not necessarily have a given disease, but may be merely seeking medical advice. The term “subject” as used herein includes all members of the animal kingdom prone to suffering from the indicated disorder. In some aspects, the subject is a mammal, and in some aspects, the subject is a human.

“Treating” or “treatment” as used herein includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration, diminishment of the reoccurrence of disease. Treatment may prevent the disease from occurring; relieve the disease's symptoms, fully or partially remove the disease's underlying cause, shorten a disease's duration, or do a combination of the above.

“Treating” and “treatment” as used herein may include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

As used herein, “topical application,” “topical administration,” and “topically administering” are used interchangeably herein and include the administration to a subject. Topical application or administering may result in the delivery of an active agent to the eye.

“Topical formulation” and “topical pharmaceutical composition” are used interchangeably herein and include a formulation that is suitable for topical application to the eye. A topical formulation may, for example, be used to confer a therapeutic benefit to its user.

As used herein, the phrase “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids.

Non-limiting examples of base salts include ammonium salts; alkali metal salts, such as sodium and potassium salts; alkaline earth metal salts, such as calcium and magnesium salts; salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others.

The compositions can be administered prior to, concurrently with, and/or after the development of ptosis. The compositions may be administered between 1 and 7 days a week, for a period of time necessary to achieve the desired results, which may be several days to several months or continuously. The compositions can be administered once or several times (2, 3, 4, or more times) a day depending on the desired effect. In certain embodiments, the compositions can be administered every 1, 2, 3, 4, 5, 6, or 7 days. In another embodiment, the compositions can be administered one or more times every 1, 2, 3, or 4 weeks. The administration can be on a monthly or bi-monthly basis. Further, the compositions can be administered for 1, 2, 3, 6, 9, or 12 months or continuously. In certain embodiments, the compositions can be administered on an ongoing basis to maintain a desired result. The compositions can be administered once a day, twice a day, three times a day and up to four times a day.

As used herein, “carrier,” “inert carrier,” and “acceptable carrier” may be used interchangeably and refer to a carrier which may be combined with the presently disclosed compounds in order to provide a desired composition. In accordance with one embodiment, the composition includes a ophthalmologically acceptable vehicle or carrier. The vehicle, which may be employed for preparing compositions may comprise, for example, aqueous solutions, dispersions, emulsions, suspensions, or ointments.

In accordance with the disclosure, the ophthalmic composition of the present invention can optionally include one or more agents such as, without limitation, emulsifying agents, wetting agents, tonicity adjusters, preservatives, buffers antioxidants and flavonoids. Tonicity adjustors useful in a pharmaceutical composition of the present disclosure include, but are not limited to, salts such as sodium acetate, sodium chloride, potassium chloride, mannitol or glycerin and other pharmaceutically acceptable tonicity adjusters. Preservatives useful in the pharmaceutical compositions described herein include, without limitation, benzalkonium chloride, chlorobutanol, thimerosal, phenyl mercuric acetate, Purite® and phenyl mercuric nitrate. Various buffers and means for adjusting pH can be used to prepare a pharmaceutical composition, including but not limited to, acetate buffers, citrate buffers, phosphate buffers and borate buffers. Similarly, antioxidants useful in pharmaceutical compositions are well known in the art and include for example, sodium metabisulfite, sodium thiosulfate, acetylcysteine, butylated hydroxyanisole and butylated hydroxytoluene. Flavonoids are compounds found in plants that are well known to have diverse beneficial biochemical and antioxidant effects. Subcategories of flavonoids include: flavones, flavonols, flavanonse and flavanonols. Examples of flavonoids include: luteolin, apigenin, tangeritin, quercetin, kaempferol, myricetin, fisetin, isorhamnetin, pachypodol, rhamnazin, hesperetin, naringenin, eriodictyol, homoeriodictyol, taxifolin, dihydroquercetin, dihydrokaempferol, tannic acid, tannis, condensed tannis, and hydrolysable tannis.

The compounds described herein may be administered at least in the minimum dose necessary to achieve the desired therapeutic effect. Generally, such doses will be in the range of about 1 ml/day to about 100 ml/day; more preferably in the range of about 10 ml/day to about 500 ml/day. In another example embodiment, the compound or active agents may be present in a composition or formulation in a range of about 0.0001 mg/kg/day to about 100 mg/kg/day or about 0.01mg/kg/day to about 100 mg/kg/day. However, the actual amount of the compound to be administered in any given case will be determined by a physician taking into account the relevant circumstances, such as the age and weight of a patient, patient's general physical condition, severity of the skin blemish, and route of administration. In some instances, dosing is evaluated on a case-by-case basis. Compositions may be applied topically, by injection, or in an ocular implant.

Additionally, compositions may be designed to delay release of the compound over a given period of time, or to carefully control the amount of compound released at a given time during the course of treatment.

The pH of the disclosed compositions can be about 3 to about 8.0, or about 6.5 to about 7.5. In certain embodiments, the pH of the formulation is about 7.0 to about 7.4 or about 7.1 to about 7.3.

Any reference made to patents and printed publications throughout this specification is individually incorporated herein by reference in its entirety.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### FORMULATIONS

The present invention is comprised of formulations and methods for the treatment of ptosis. Formulations include at least the active agents oxymetazoline and pilocarpine.

Oxymetazoline, C₁₆H₂₄N₂O, has the following structure:

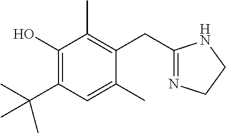 [SMILES: Cc1cc(C(C)(C)C)c(O)c(C)c1CC1=NCCN1]

Oxymetazoline may be used in a salt form, such as oxymetazoline HCl:

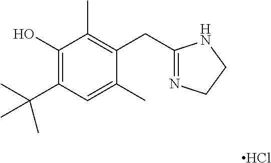 [SMILES: Cc1cc(C(C)(C)C)c(O)c(C)c1CC1=NCCN1 ; Cl]

Other salts of oxymetazoline include, but are not limited to oxymetazoline hydrobromide, oxymetazoline hydrofluoride, oxymetazoline sulphate, oxymetazoline hydroiodide, oxymetazoline nitrate, oxymetazoline citrate, oxymetazoline formate, oxymetazoline acetate, oxymetazoline tartrate and oxymetazoline fumarate.

Pilocarpine, C₁₁H₁₆N₂O₂, has the following structure:

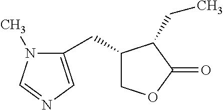

Pilocarpine may be used in a salt form:

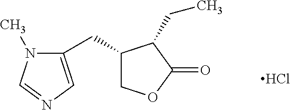

Other possible salts include, but are not limited to, pilocarpine mononitrate, pilocarpine nitrate, pilocarpine muriate and pilocarpine monohydrate.

In one embodiment, the formulation is about 0.0125% w/v oxymetazoline and about 0.25% w/v pilocarpine. In one embodiment, the formulation is about 0.0125% w/v oxymetazoline and about 0.5% w/v pilocarpine. In one embodiment, the formulation is about 0.0125% w/v oxymetazoline and about 1% w/v pilocarpine. In one embodiment, the formulation is about 0.0125% w/v oxymetazoline and about 1.5% w/v pilocarpine. In one embodiment, the formulation is about 0.0125% w/v oxymetazoline and about 2% w/v pilocarpine. In one embodiment, the formulation is 0.0125% w/v oxymetazoline and 0.25% w/v pilocarpine. In one embodiment, the formulation is 0.0125% w/v oxymetazoline and 0.5% w/v pilocarpine. In one embodiment, the formulation is 0.0125% w/v oxymetazoline and 1% w/v pilocarpine. In one embodiment, the formulation is 0.0125% w/v oxymetazoline and 1.5% w/v pilocarpine. In one embodiment, the formulation is 0.0125% w/v oxymetazoline and 2% w/v pilocarpine.

In one embodiment, the formulation is about 0.025% w/v oxymetazoline and about 0.25% w/v pilocarpine. In one embodiment, the formulation is about 0.025% w/v oxymetazoline and about 0.5% w/v pilocarpine. In one embodiment, the formulation is about 0.025% w/v oxymetazoline and about 1% w/v pilocarpine. In one embodiment, the formulation is about 0.025% w/v oxymetazoline and about 1.5% w/v pilocarpine. In one embodiment, the formulation is about 0.025% w/v oxymetazoline and about 2% w/v pilocarpine. In one embodiment, the formulation is 0.025% w/v oxymetazoline and 0.25% w/v pilocarpine. In one embodiment, the formulation is 0.025% w/v oxymetazoline and 0.5% w/v pilocarpine. In one embodiment, the formulation is 0.025% w/v oxymetazoline and 1% w/v pilocarpine. In one embodiment, the formulation is 0.025% w/v oxymetazoline and 1.5% w/v pilocarpine. In one embodiment, the formulation is 0.025% w/v oxymetazoline and 2% w/v pilocarpine.

In one embodiment, the formulation is about 0.05% w/v oxymetazoline and about 0.25% w/v pilocarpine. In one embodiment, the formulation is about 0.05% w/v oxymetazoline and about 0.5% w/v pilocarpine. In one embodiment, the formulation is about 0.05% w/v oxymetazoline and about 1% w/v pilocarpine. In one embodiment, the formulation is about 0.05% w/v oxymetazoline and about 1.5% w/v pilocarpine. In one embodiment, the formulation is about 0.05% w/v oxymetazoline and about 2% w/v pilocarpine. In one embodiment, the formulation is 0.05% w/v oxymetazoline and 0.25% w/v pilocarpine. In one embodiment, the formulation is 0.05% w/v oxymetazoline and 0.5% w/v pilocarpine. In one embodiment, the formulation is 0.05% w/v oxymetazoline and 1% w/v pilocarpine. In one embodiment, the formulation is 0.05% w/v oxymetazoline and 1.5% w/v pilocarpine. In one embodiment, the formulation is 0.05% w/v oxymetazoline and 2% w/v pilocarpine.

In one embodiment, the formulation is about 0.1% w/v oxymetazoline and about 0.25% w/v pilocarpine. In one embodiment, the formulation is about 0.1% w/v oxymetazoline and about 0.5% w/v pilocarpine. In one embodiment, the formulation is about 0.1% w/v oxymetazoline and about 1% w/v pilocarpine. In one embodiment, the formulation is about 0.1% w/v oxymetazoline and about 1.5% w/v pilocarpine. In one embodiment, the formulation is about 0.1% w/v oxymetazoline and about 2% w/v pilocarpine. In one embodiment, the formulation is 0.1% w/v oxymetazoline and 0.25% w/v pilocarpine. In one embodiment, the formulation is 0.1% w/v oxymetazoline and 0.5% w/v pilocarpine. In one embodiment, the formulation is 0.1% w/v oxymetazoline and 1% w/v pilocarpine. In one embodiment, the formulation is 0.1% w/v oxymetazoline and 1.5% w/v pilocarpine. In one embodiment, the formulation is 0.1% w/v oxymetazoline and 2% w/v pilocarpine.

In one embodiment, the formulation is about 0.125% w/v oxymetazoline and about 0.25% w/v pilocarpine. In one embodiment, the formulation is about 0.125% w/v oxymetazoline and about 0.5% w/v pilocarpine. In one embodiment, the formulation is about 0.125% w/v oxymetazoline and about 1% w/v pilocarpine. In one embodiment, the formulation is about 0.125% w/v oxymetazoline and about 1.5% w/v pilocarpine. In one embodiment, the formulation is about 0.125% w/v oxymetazoline and about 2% w/v pilocarpine. In one embodiment, the formulation is 0.125% w/v oxymetazoline and 0.25% w/v pilocarpine. In one embodiment, the formulation is 0.125% w/v oxymetazoline and 0.5% w/v pilocarpine. In one embodiment, the formulation is 0.125% w/v oxymetazoline and 1% w/v pilocarpine. In one embodiment, the formulation is 0.125% w/v oxymetazoline and 1.5% w/v pilocarpine. In one embodiment, the formulation is 0.125% w/v oxymetazoline and 2% w/v pilocarpine.

In one embodiment, the formulation is about 0.2% w/v oxymetazoline and about 0.25% w/v pilocarpine. In one embodiment, the formulation is about 0.2% w/v oxymetazoline and about 0.5% w/v pilocarpine. In one embodiment, the formulation is about 0.2% w/v oxymetazoline and about 1% w/v pilocarpine. In one embodiment, the formulation is about 0.2% w/v oxymetazoline and about 1.5% w/v pilocarpine. In one embodiment, the formulation is about 0.2% w/v oxymetazoline and about 2% w/v pilocarpine. In one embodiment, the formulation is 0.2% w/v oxymetazoline and 0.25% w/v pilocarpine. In one embodiment, the formulation is 0.2% w/v oxymetazoline and 0.5% w/v pilocarpine. In one embodiment, the formulation is 0.2% w/v oxymetazoline and 1% w/v pilocarpine. In one embodiment, the formulation is 0.2% w/v oxymetazoline and 1.5% w/v pilocarpine. In one embodiment, the formulation is 0.2% w/v oxymetazoline and 2% w/v pilocarpine.

In some embodiments, oxymetazoline is present from a percent w/v of about 0.01 to about 0.5, from about 0.01 to about 0.2, from about 0.01 to about 0.15, from about 0.0125 to about 0.15, from about 0.0125 to about 0.125, from about 0.02 to about 0.15, from about 0.03 to about 0.15, from about 0.04 to about 0.15, from about 0.05 to about 0.15, from about 0.06 to about 0.15, from about 0.07 to about 0.15, from about 0.08 to about 0.15, from about 0.09 to about 0.15, from about 0.1 to about 0.15, from about 0.11 to about 0.15, from about 0.115 to about 0.15, from about 0.120 to about 0.15, and from about 0.125 to about 0.15, from about 0.125 to about 0.145, from about 0.125 to about 0.14, from about 0.02 to about 0.08, from about 0.03 to about 0.08, from about 0.04 to about 0.08, from about 0.05 to about 0.08, from about 0.06 to about 0.08, from about 0.07 to about 0.08, from about 0.02 to about 0.07, from about 0.03 to about 0.07, from about 0.04 to about 0.07, from about 0.05 to about 0.07, from about 0.06 to about 0.07, from about 0.02 to about 0.06, from about 0.03 to about 0.06, from about 0.04 to about 0.06, from about 0.05 to about 0.06, from about 0.02 to about 0.05, from about 0.03 to about 0.05, from about 0.04 to about 0.05, from about 0.02 to about 0.04, from about 0.03 to about 0.04, or from about 0.02 to about 0.03% (w/w). In some embodiments, the oxymetazoline is present at about 0.01, 0.012, 0.0125, 0.013, 0.014, 0.15, 0.02, 0.25, 0.03, 0.35, 0.04, 0.45, 0.05, 0.06, 0.07, 0.075, 0.08, 0.09, or 0.1, 0.11, 0.12, 0.121, 0.122, 0.125, 0.13, 0.135, 0.140, 0.145, 0.150, 0.155. 0.160, 0.165, 0.170, 0.175, 0.180., 0.185, 0.190, 0.195, 0.2, 0.25, 0.30, 0.35, 0.4, 0.45 and 0.5 (% w/v).

In some embodiments pilocarpine is present in from a percent w/v of about 0.01 to about 5, about 0.01 to about 2, about 0.01 to about 1.5, about 0.01 to about 1.0, about 0.01 to about 0.5, about 0.01 to about 0.25, from about 0.02 to about 0.5, from about 0.03 to about 0.5, from about 0.04 to about 0.5, from about 0.05 to about 0.5, from about 0.06 to about 0.5, from about 0.07 to about 0.5, from about 0.08 to about 0.5, from about 0.09 to about 0.5, from about 0.1 to about 0.5, from about 0.15 to about 0.5, from about 0.2 to about 0.5, from about 0.3 to about 0.5, and from about 0.4 to about 0.5, from about 0.5 to about 2, from about 0.5 to about 1.5, from about 1 to about 2, from about 1 to about 1.5, or is present at about 0.01, 0.02, 0.03, 0.04, 0.05, 0.06, 0.07, 0.08, 0.09, or 0.1, 0.11, 0.12, 0.121, 0.122, 0.125, 0.13, 0.135, 0.140, 0.145, 0.150, 0.155. 0.160, 0.165, 0.170, 0.175, 0.180, 0.185, 0.190, 0.195, 0.2, 0.25, 0.30, 0.35, 0.4, 0.45, 0.5, 0.6, 0.7, 0.8, 0.9, 1.0, 1.1, 1.2, 1.25, 1.3, 1.35, 1.4, 1.45, 1.5, 1.6, 1.7, 1.75, 1.8, 1.9, 2.0, 2.2, 2.4, 2.5, 2.6, 2.7, 2.8, 2.9, 3.0, 3.25, 3.5, 3.75, 4, 4.25, 4.5, 4.75, 5 (% w/v).

Combinations of pilocarpine and oxymetazoline, which may be in their salt forms, are shown in Table 1. Each combination of pilocarpine and oxymetazoline is provided by a formulation number in Table I.

TABLE 1

| | Pilocarpine % w/v | | | | | | | | | | |
|---|---|---|---|---|---|---|---|---|---|---|---|
| Oxymetazoline % w/v | 1.0% | 1.05% | 1.1% | 1.15% | 1.20% | 1.25% | 1.30% | 1.35% | 1.40% | 1.45% | 1.50% |
| 0.05% | 1 | 25 | 49 | 73 | 97 | 121 | 145 | 169 | 193 | 217 | 241 |
| 0.06% | 2 | 26 | 50 | 74 | 98 | 122 | 146 | 170 | 194 | 218 | 242 |
| 0.07% | 3 | 27 | 51 | 75 | 99 | 123 | 147 | 171 | 195 | 219 | 243 |
| 0.08% | 4 | 28 | 52 | 76 | 100 | 124 | 148 | 172 | 196 | 220 | 244 |
| 0.09% | 5 | 29 | 53 | 77 | 101 | 125 | 149 | 173 | 197 | 221 | 245 |
| 0.10% | 6 | 30 | 54 | 78 | 102 | 126 | 150 | 174 | 198 | 222 | 246 |
| 0.105% | 7 | 31 | 55 | 79 | 103 | 127 | 151 | 175 | 199 | 223 | 247 |
| 0.11% | 8 | 32 | 56 | 80 | 104 | 128 | 152 | 176 | 200 | 224 | 248 |
| 0.115% | 9 | 33 | 57 | 81 | 105 | 129 | 153 | 177 | 201 | 225 | 249 |
| 0.120% | 10 | 34 | 58 | 82 | 106 | 130 | 154 | 178 | 202 | 226 | 250 |
| 0.125% | 11 | 35 | 59 | 83 | 107 | 131 | 155 | 179 | 203 | 227 | 251 |
| 0.130% | 12 | 36 | 60 | 84 | 108 | 132 | 156 | 180 | 204 | 228 | 252 |
| 0.135% | 13 | 37 | 61 | 85 | 109 | 133 | 157 | 181 | 205 | 229 | 253 |
| 0.140% | 14 | 38 | 62 | 86 | 110 | 134 | 158 | 182 | 206 | 230 | 254 |
| 0.145% | 15 | 39 | 63 | 87 | 111 | 135 | 159 | 183 | 207 | 231 | 255 |
| 0.150% | 16 | 40 | 64 | 88 | 112 | 136 | 160 | 184 | 208 | 232 | 256 |
| 0.155% | 17 | 41 | 65 | 89 | 113 | 137 | 161 | 185 | 209 | 233 | 257 |
| 0.160% | 18 | 42 | 66 | 90 | 114 | 138 | 162 | 186 | 210 | 234 | 258 |
| 0.165% | 19 | 43 | 67 | 91 | 115 | 139 | 163 | 187 | 211 | 235 | 259 |
| 0.170% | 20 | 44 | 68 | 92 | 116 | 140 | 164 | 188 | 212 | 236 | 260 |
| 0.175% | 21 | 45 | 69 | 93 | 117 | 141 | 165 | 189 | 213 | 237 | 261 |
| 0.180% | 22 | 46 | 70 | 94 | 118 | 142 | 166 | 190 | 214 | 238 | 262 |
| 0.185% | 23 | 47 | 71 | 95 | 119 | 143 | 167 | 191 | 215 | 239 | 263 |
| 0.190% | 24 | 48 | 72 | 96 | 120 | 144 | 168 | 192 | 216 | 240 | 264 |

Certain vehicle formulations (other than active agents) are disclosed in Tables II and III:

TABLE II
Vehicles

| | Vehicle Formulations | | | | | | | | | | |
|---|---|---|---|---|---|---|---|---|---|---|---|
| Ingredient % w/v | 1 | 2 | 3 | 4 | 5 | 6 | 7 | 8 | 9 | 10 | 11 |
| Active agents | | | | | | | | | | | |
| EDTA | 0.01 | 0.01 | 0.015 | — | 0.02 | 0.015 | — | 0.01 | 0.015 | 0.03 | 0.02 |
| NaCl | 0.1 | 0.05 | 0.15 | 0.1 | 0.05 | 0.15 | 0.1 | — | 0.1 | — | 0.05 |
| Mannitol | — | — | 2.0 | 5.0 | — | — | 2.0 | — | 5.0 | — | 111 |
| Glycerin | 10.0 | 5.0 | — | — | 10 | 5 | — | 10 | 5 | 10 | — |
| BAK | — | 0.2 | 0.15 | 0.2 | 0.2 | — | 0.2 | 0.2 | — | — | 0.2 |
| Polysorbate 20 | 0.1 | — | 1.0 | 0.5 | — | 0.2 | 0.5 | 0.1 | 1.0 | 1.0 | — |
| Polysorbate 40 | — | 0.1 | — | — | — | 0.3 | — | — | — | — | — |
| Carboxymethyl | — | — | — | — | 0.5 | — | — | — | — | — | 1.0 |
| cellulose | | | | | | | | | | | |
| Ascorbic Acid | 0.1 | 0.1 | 0.1 | 0.1 | 0.1 | — | 0.1 | 0.1 | 0.1 | 0.1 | 0.1 |
| NaOH/HCl (pH) | 6.8 | 7.0 | 6.5 | 7.0 | 7.1 | 6.4 | 6.5 | 7.1 | 6.8 | 6.9 | 6.9 |
| Purified | Q.S. | Q.S. | Q.S. | Q.S. | Q.S. | Q.S. | Q.S. | Q.S. | Q.S. | Q.S. | Q.S. |
| Water/WFI | | | | | | | | | | | |

TABLE III
Vehicle

| Ingredient | Function | Concentration (% w/v) |
|---|---|---|
| Carboxymethycellulose | Thickening Agent | 0.325% |
| Sodium (Med. Viscosity) | | |
| Carboxymethycellulose | Thickening Agent | 0.175% |
| Sodium (High Viscosity) | | |
| NaCl | Tonicity Agent | 0.7% |
| Sodium Citrate | Buffer | 0.2% |
| Dihydrate | | |
| Sodium Hydroxide (1N) | pH adjustment | Adjust to pH 6.8 |
| Hydrochloric Acid (1N) | pH adjustment | Adjust to pH 6.8 |
| Purified Water | Vehicle | Q.S. |

It is intended that any of the numbered formulations of the active agents in Table I can be matched with any of the vehicle formulations of Table II or Table III.

The formulations of the present invention are suitable for topical administration. In certain embodiments, the composition is a solution, a suspension, an emulsion, an ointment, a gel, or a solid insert. The disclosure includes microemulsions and reverse emulsions (i.e., water in oil). Microemulsions are clear, stable, isotropic liquid mixtures of oil, water and a surfactant, frequently in combination with a cosurfactant.

### EXAMPLES

Non-limiting examples of compositions in accordance with the present invention are as follows.

### Example I

A 34 year old Caucasian male suffering from mild acquired ptosis after experiencing trauma to the levator muscle in his right eye. As a result, the patient experiences slight drooping of the right upper eyelid. While not functionally debilitating or causing any medical issues, the slight drooping of the right eyelid causes the patient concern as to his appearance. After being prescribed a topical formulation comprising 0.125% w/v oxymetazoline and 1.0% w/v pilocarpine (Formula 11 in Table 1 in formulation vehicle 4 in Table II), the patient applies two drops daily with one drop in the morning and one drop in twelve hours later in the evening in his right eye. After two days, the patient will notice an improvement in the right eyelid which retracts upward. After seven days of use, the right eyelid will be approximately equal to the left eyelid in the amount of retraction.

### Example II

A 60 year old Asian female suffers from congenital severe ptosis in both eyes which has worsened with age. The patient has a levator function of approximately 5.5 mm. The patient undergoes levator muscle resection and brow/frontalis suspension and a autogenous fascia lata grafts. While the surgery is successful for the left eye, the right eye is under corrected resulting in a lack of symmetry between the eyes. The patient is prescribed topical application of a twice daily dose of a 0.15% w/v oxymetazoline/1.25% w/v pilocarpine formulation (formulation No. 136 in Table 1) with a vehicle as taught in formulation I in Table II. After one day of use, the patient's left upper eyelid will lift approximately 2 mm and approximate symmetry between both eyes is achieved and will be maintained as long as the patient continues the twice daily application of the formulation.

### Example III

A 55 year old African American male suffers from myogenic ptosis in both eyes and has previously undergone Muller's muscle conjunctival resection (resectioning of Muller's muscle and of the conjunctiva) via a posterior approach. However, the patient's myogenic ptosis is progressive and worsens despite the surgical correction. The patient also suffers from dry-eye syndrome, and due to decreased tarsal stability and fewer accessory lacrimal glands postoperatively, the patient has less basal tear secretion. Consequently, the patient is not a candidate for further surgery. Instead of surgery, the patient is prescribed a once daily regimen of 0.135% w/v oxymetazoline/1.5% w/v pilocarpine formulation (formulation No. 253 of Table 1) and a vehicle as taught in vehicle of Table III. After five days of use, both upper eyelids will retract approximately 2.5 mm and the patient will no longer require surgery.

### Example IV

A 72 year old Caucasian female who has suffered a stroke has complete right side ptosis and can no longer see out of her right eye. She is believed to suffer from localized myogenic dysgenesis from deterioration of the levator muscle. The levator muscle is unable to completely contract and relax and a result her right eyelid. The patient applies 0.15% w/v oxymetazoline/1.5% w/v pilocarpine (Formula 256 of Table I in vehicle 5 of Table II) and after two days her upper eyelid begins to retract and after one week the patient's eyelid will normally retract as long as the patient applies the formulation.

### Example V

A 32 year old Hispanic male suffers from Homer's syndrome with neural damage to the sympathetic trunk of the right side of his face. The patient suffers from a constricted pupil and upside-down ptosis characterized in slight elevation of the lower right eyelid. The patient also complains of poor vision at night. The patient applies twice daily several drops of 0.10% w/v oxymetazoline/1.0% w/v pilocarpine (Formula 6 in Table I) in vehicle 3 of Table II. The patient will notice immediate results including retraction of the lower right eyelid and improving night vision.

### Example VI

A 41 year old Caucasian female is suffering from myasthenia gravis and as a result has myogenic ptosis in her left eye. The patient has had multiple surgeries to correct her ptosis of the left eye but has experienced a recurrence. The patient administers drops of 0.135% w/v oxymetazoline and 1.1% w/v pilocarpine (Formulation 61 of Table I) in vehicle 5 of Table II three times a day (“TID”). After two weeks of administration, the patient's left eyelid will retract normally and no longer suffers from myogenic ptosis.

### Example VII

A 53 year old African American male has severe ptosis (>4 mm) in his right eye with little or no levator function which is so severe he can no longer see out the eye. The patient undergoes a brow/frontalis suspension procedure with collagen injections in the brow. While the surgery successfully improves levator function, the patient still is experiencing moderate ptosis of 3-4 mm. The patient self-administers twice daily (“BID) three drops of a 0.125% w/v oxymetazoline/1.25% w/v pilocarpine formulation (Formulation 131 of Table 1 and vehicle formulation 2 of Table II) and after several days, the moderate ptosis improves to minimal ptosis (1-2 mm) and in two weeks of continuous administration, ptosis symptoms will disappear altogether.

### Example VIII

A four year old Caucasian male pediatric patient has mild congenital myogenic ptosis in his left eye and is believed to have occurred due to faulty development of the levator muscle. The condition is progressive and worsening. The patient begins developing amblyopia (“lazy-eye”) as a result of not being able to see properly out of his left eye. Rather than undergo surgical correction, the patient receives a 0.125% w/v oxymetazoline/1.0% w/v pilocarpine formulation 11 in Table I and vehicle 9 in Table II) once a day. After several days, the patient's upper eyelid begins to retract normally and no longer exhibits ptosis and the amblyopia gradually improves.

### Example IX

A 71 year old Caucasian female has difficulty driving at night due particularly in low ambient light environments. The patient self administers three drops of 0.125% w/v oxymetazoline/1.0% w/v pilocarpine formulation 11 in Table I and vehicle 10 in Table II reducing pupil dilation thereby improving her vision in low ambient light environments.

### Example X

A 51 year old Asian male presents with difficulty in seeing in low ambient light conditions due to an abnormally dilated pupils. The patient self administers two drops of 0.15% w/v oxymetazoline/1.5% w/v pilocarpine formulation (formulation 254 in Table I and vehicle formulation 7 in Table II) and the patient's pupil diameter in low ambient light and experiences improved vision.

What is claimed:

1. A method of treating ptosis in a patient suffering therefrom comprising administering a combination of pilocarpine and oxymetazoline.

2. The method of claim 1 wherein pilocarpine and oxymetazoline are administered in a single formulation.

3. The method of claim 2 wherein the composition is administered topically to the ptotic eye.

4. The method of treating ptosis comprising administering to a subject in need thereof a composition comprising a therapeutically effective amount of a composition comprising pilocarpine and oxymetazoline.

5. The method of claim 1 wherein the composition comprises about 0.125% w/v oxymetazoline and about 1.0% w/v pilocarpine.

6. The method of claim 1 wherein the composition comprises about 0.0125-0.2% w/v oxymetazoline and about 0.05%-1.5% w/v pilocarpine.

7. The method of claim 5 wherein the composition is dosed at least once a day.

8. The method of claim 7, wherein the composition is dosed twice a day.

9. The method of claim 7, wherein the administration of the composition results in reduction of the symptoms of ptosis.

10. The method of claim 1 wherein oxymetazoline and pilocarpine are administered concurrently.

11. A composition for use in treating blepharoptosis comprising oxymetazoline and pilocarpine.

12. The composition of claim 11 wherein the composition comprises from about 0.01-0.2% w/v oxymetazoline and about 0.05-1.5% w/v pilocarpine.

13. The composition of claim 12 wherein the composition comprises from about 0.0125-0.125% w/v oxymetazoline and about 0.5-1.5% w/v pilocarpine.

14. The composition of claim 13 wherein the composition comprises about 0.125% w/v oxymetazoline and about 1.0% w/v pilocarpine and at least one excipient from Table II.

15. The composition of claim 14 wherein the composition comprises 0.125% w/v oxymetazoline and 1.0% w/v pilocarpine.

16. The composition of claim 11 wherein the composition is preserved.

17. The composition of claim 16 wherein the preservative is benzalkonium chloride.

18. The composition of claim 11 wherein the composition is preservative free.

19. The composition of claim 11 wherein the composition is an aqueous solution formulated for topical administration.

20. The composition of claim 11 wherein the composition is in the form of an ocular implant.
